(19) United States
(12) Patent Application Publication
(10) Pub. No.: US 2025/0208240 A1
(43) Pub. Date: Jun. 26, 2025

(54) OBJECT POSITIONING DEVICE, BED, MAGNETIC RESONANCE SCANNING METHOD

(71) Applicant: Shanghai United Imaging Healthcare Co., Ltd., Shanghai (CN)

(72) Inventors: Xiangbin Fang, Shanghai (CN); Feichao Fu, Shanghai (CN); Yifeng Wang, Shanghai (CN); Junjun Qin, Shanghai (CN); Jiawen Fan, Shanghai (CN)

(21) Appl. No.: 19/001,464

(22) Filed: Dec. 25, 2024

(30) Foreign Application Priority Data
Dec. 25, 2023 (CN) 202311808265.X

(51) Int. Cl. G01R 33/34; A61B 5/00

(52) U.S. Cl. CPC G01R 33/34007; A61B 5/704

(57) ABSTRACT

An object positioning device provided by the present disclosure includes at least one coil and a positioning plate. At least a part of the at least one coil is configured to be embedded in the positioning plate.

### CROSS-REFERENCE TO RELATED APPLICATIONS

The present application claims priority to Chinese patent application No. 202311808265.X, titled “OBJECT POSITIONING DEVICE, BED, MAGNETIC RESONANCE SCANNING METHOD AND RADIATION DELIVERY METHOD”, filed on Dec. 25, 2023, the entire content of which is incorporated herein by reference.

### TECHNICAL FIELD

The present disclosure relates to the field of medical technologies, and in particular, to an object positioning device, a bed, and a magnetic resonance scanning method.

### BACKGROUND

With the development of medical technology, radiotherapy equipment has emerged, and magnetic resonance imaging can be performed by using radiotherapy equipment. In order to achieve a better effect in magnetic resonance imaging, it is necessary to ensure that a radio frequency (RF) receiving coil in the radiotherapy equipment approaches an object's body surface as closely as possible to improve the receiving efficiency of the RF receiving coil.

Therefore, how to improve the receiving efficiency of the RF receiving coil without affecting other imaging performance aspects of the radiotherapy equipment has become a technical problem that needs to be solved urgently in the current medical technology field.

### SUMMARY

Based on this, the present disclosure provides an object positioning device, a bed, a magnetic resonance scanning method, and a radiation delivery method.

In a first aspect, the present disclosure provides an object positioning device. The object positioning device includes at least one coil and a positioning plate. At least a part of the at least one coil is configured to be embedded in the positioning plate.

In an embodiment, a groove is provided on or in the positioning plate, and the at least a part of the at least one coil is fixed in the groove.

In an embodiment, a depth of the groove is less than or equal to a height of the positioning plate, and a height of the at least one coil is less than or equal to the depth of the groove.

In an embodiment, a slot is provided on or in the positioning plate, and the at least a part of the at least one coil is slidably arranged in the positioning plate through the slot.

In an embodiment, the positioning plate further includes at least one protrusion component disposed in the groove, and the at least a part of the at least one coil is arranged to surround the at least one protrusion component.

In an embodiment, the positioning plate includes a plurality of protrusion components, and the plurality of protrusion components are disposed in the groove in an array.

In an embodiment, the at least a part of the at least one coil is arranged to detachably surround the at least one protrusion component.

In an embodiment, the at least a part of the at least one coil is detachably fixed in the groove.

In an embodiment, the positioning plate further includes sliding components. The sliding components are provided on any two opposite sides of the groove, and the sliding components on both sides are connected to two ends of the at least one coil, respectively.

In an embodiment, the at least one coil includes a radio frequency receiving coil.

In an embodiment, the positioning plate includes a U-shaped coil support structure bent upward on a periphery of the positioning plate.

In an embodiment, the object positioning device further includes a coil plate. The coil plate is provided with a plurality of coils with holes, the plurality of coils are arranged in an array, and the hole of each coil is configured to snap with the corresponding protrusion component correspondingly.

In an embodiment, the coil plate is further provided with signal transmission lines for the coils, and the signal transmission lines are configured to transmit signals of the coils.

In a second aspect, the present disclosure further provides a bed. The bed includes an object positioning device, an isolation pad and a bed body. The object positioning device is arranged on the bed body, and the isolation pad is arranged on the object positioning device. The object positioning device includes at least one coil and a positioning plate, and at least a part of the at least one coil is configured to be embedded in the positioning plate.

In an embodiment, the bed further includes at least one support pad. The at least one support pad and the object positioning device are arranged substantially in a same plane, the at least one support pad is arranged on the bed body, and the isolation pad is arranged on the object positioning device and the at least one support pad.

In an embodiment, the bed further includes abed board. The object positioning device is embedded in the bed board, the bed board is arranged on the bed body, and the isolation pad is arranged on the bed board.

In an embodiment, the bed board has a concave, and the object positioning device is embedded within the concave.

In an embodiment, the bed further includes a bed board. The bed board is arranged on the bed body, the object positioning device is arranged on the bed board, and the isolation pad is arranged on the object positioning device.

In an embodiment, a groove is provided on or in the positioning plate, and the at least a part of the at least one coil is fixed in the groove.

In an embodiment, the positioning plate further includes at least one protrusion component disposed in the groove, and the at least a part of the at least one coil is arranged to surround the at least one protrusion component.

In a third aspect, the present disclosure further provides a magnetic resonance scanning method. The magnetic resonance scanning method includes: placing an object on a bed, the bed including an object positioning device, an isolation pad and a bed body, the object positioning device being arranged on the bed body, the isolation pad being arranged on the object positioning device, the object positioning device including at least one coil and a positioning plate, and at least a part of the at least one coil being configured to be embedded in the positioning plate; and performing a magnetic resonance scan on the object.

### BRIEF DESCRIPTION OF THE DRAWINGS

In order to describe the technical solutions of the embodiments of the present disclosure or the conventional art more clearly, the accompanying drawings required for describing the embodiments or for describing the conventional art will be briefly introduced as follows. Apparently, the accompanying drawings, in the following description, illustrate merely some embodiments of the present disclosure, for a person of ordinary skill in the art, other drawings can also be obtained according to these accompanying drawings without making any creative efforts.

FIG. 1 is a schematic diagram illustrating a structure of an object positioning device in an embodiment of the present disclosure.

FIG. 2 is a schematic diagram illustrating a structure of an object positioning device including a protrusion component in an embodiment of the present disclosure.

FIG. 3 is a schematic diagram illustrating a structure of an object positioning device including a plurality of protrusion components in an embodiment of the present disclosure.

FIG. 4 is a schematic diagram illustrating a structure of a plurality of coils with holes in an embodiment of the present disclosure.

FIG. 5 is a schematic diagram illustrating a structure of an object positioning device including sliding components in an embodiment of the present disclosure.

FIG. 6 is a schematic diagram illustrating a structure of a bed in an embodiment of the present disclosure.

FIG. 7 is a schematic diagram illustrating a structure of a bed including a support pad in an embodiment of the present disclosure.

FIG. 8 is a schematic diagram illustrating a structure of a bed including a plurality of support pads in an embodiment of the present disclosure.

FIG. 9 is a schematic diagram illustrating a structure of a bed including a bed board in an embodiment of the present disclosure.

FIG. 10 is a schematic diagram of a structure of a bed board in an embodiment of the present disclosure.

FIG. 11 is a flow diagram illustrating a magnetic resonance scanning method in an embodiment of the present disclosure.

FIG. 12 is a flow diagram illustrating a radiation delivery method in an embodiment of the present disclosure.

### DETAILED DESCRIPTION OF THE EMBODIMENTS

In order to make the objectives, technical solutions and advantages of the present disclosure more clearly understood, the present disclosure will be further described in detail with the accompanying drawings and embodiments. It should be understood that the specific embodiments described herein are only used to explain the present disclosure and not to limit the present disclosure.

Unless otherwise defined, all technical and scientific terms used herein have the same meaning as commonly understood by a person of ordinary skill in the art to which the present disclosure belongs. The terms used herein in the application are for the purpose of describing specific embodiments only, and are not intended to limit the present disclosure. The terms “including” and “having” and any variations thereof in the specification and claims and the above descriptions of the drawings of the present disclosure are intended to cover non-exclusive inclusion.

In the description of the present disclosure, it should be understood that the orientation or position relationship indicated by the terms “center”, “longitudinal”, “transverse”, “length”, “width”, “thickness”, “upper”, “lower”, “front”, “back”, “left”, “right”, “vertical”, “horizontal”, “top”, “bottom”, “inner”, “outer”, “clockwise”, “counterclockwise”, “axial”, “radial”, “circumferential”, etc. are based on the orientation or position relationship shown in the accompanying drawings, and are merely intended to facilitate the description of the present disclosure and simplify the description, rather than indicating or implying that the indicated device or element must have a particular orientation or be constructed and operated in a particular orientation, and therefore are not to be interpreted as limiting the present disclosure.

In addition, the terms such as “first” and “second” are used for descriptive purposes only, and should not be understood as indicating or implying relative importance or implicitly indicating the quantity of the technical features indicated. Thus, the features described with “first” and “second”, etc., may explicitly or implicitly include at least one of these features. In the description of the present disclosure, if the term “plurality” is used, it means at least two, such as two, three, etc., unless otherwise clearly and specifically defined.

In the present disclosure, unless otherwise clearly specified and limited, the terms “mounted”, “coupling”, “connection”, “fixation”, etc., should be understood in a broad sense, for example, it may be a fixed connection, a detachable connection, or integration. It may be a mechanical connection or an electrical connection. It may be a direct connection or an indirect connection through an intermediate medium. It may be an internal connection between two elements or an interaction relationship between the two elements, unless otherwise clearly defined. Those of ordinary skill in the art can understand the specific meanings of the above terms in the present disclosure according to specific situations.

In the present disclosure, unless otherwise clearly specified and limited, a first feature being “on” or “under” a second feature may mean that the first and second features are in direct contact, or the first and second features are in indirect contact through an intermediate medium. Moreover, the first feature being “on”, “above”, or “over” the second feature may mean that the first feature is directly above or obliquely above the second feature, or simply means that the horizontal height of the first feature is greater than that of the second feature. The first feature being “under”, “beneath” and “below” the second feature may mean that the first feature is directly below or obliquely below the second feature, or simply means that the horizontal height of the first feature is less than the that of second feature.

It should be noted that when an element is referred to as being “fixed on” or “arranged on” another element, it may be directly on the other element or there may be an intervening element. When an element is referred to as being “connected to” another element, it may be directly connected to the other element or there may also be an intermedium element. The terms “vertical”, “horizontal”, “upper”, “lower”, “left”, “right” and similar expressions used in this disclosure are for the purpose of illustration only and are not meant to be the only implementation methods.

Reference herein to “embodiments” means that particular features, structures, or characteristics described with reference to the embodiments can be included in at least one embodiment of the present disclosure. The occurrences of this phrase at various places in the specification do not necessarily refer to the same embodiment, nor are separate or alternative embodiments mutually exclusive of other embodiments. It is understood explicitly and implicitly by those skilled in the art that the embodiments described herein can be combined with other embodiments.

With the development of medical technology, radiotherapy equipment has emerged. Due to the excellent soft tissue imaging capabilities and characteristics of no ionizing radiation, radiation therapy equipment can be used for magnetic resonance imaging (MRI). The radiotherapy equipment includes an RF receiving coil, which is configured to receive small magnetic resonance signals. The closer the RF receiving coil is to a target imaging region on a body surface of an object, the better the signal-to-noise ratio of the RF receiving coil is, and thus the better the effect of magnetic resonance imaging is. Therefore, in order to achieve a better effect in magnetic resonance imaging, it is necessary to ensure that the RF receiving coil in the radiotherapy equipment approaches the body surface of the object as closely as possible to improve a receiving efficiency of the RF receiving coil. However, due to the special nature of magnetic resonance-guided radiotherapy equipment, positioning components such as a positioning plate and a vacuum pad are generally required to assist with the placement of the object, so that the object can maintain a correct and stable position during the magnetic resonance imaging. Furthermore, since the RF receiving coil has a certain degree of flexibility, the RF receiving coil will be deformed when being compressed by the body surface of the object, so it is necessary to customize a coil support pad for the RF receiving coil. Since there are components such as the positioning plate, the vacuum pad, the coil support pad, etc. between the RF receiving coil and the object in the conventional method, there is a large distance between the RF receiving coil and the target imaging region on the body surface of the object in the conventional method, resulting in poor imaging quality of magnetic resonance imaging.

At present, in order to address the problem of poor imaging quality of magnetic resonance imaging caused by the large distance between the RF receiving coil and the target imaging region on the body surface of the object, a size of a single-channel RF receiving coil may be enlarged to obtain an ability to receive magnetic resonance signals at a greater depth, thereby improving the signal-to-noise ratio of the RF receiving coil. Although the above method can improve the signal-to-noise ratio of the RF receiving coil to a certain extent, the above method greatly affects other imaging performance aspects of the radiotherapy equipment. Exemplarily, the above method not only affects an accelerated imaging performance of parallel acquisition during magnetic resonance imaging, but also further raises the body of the object, thereby reducing the space for the object in a cavity of the radiotherapy equipment. Therefore, how to improve the receiving efficiency of the RF receiving coil without affecting other imaging performance aspects of the radiotherapy equipment has become a technical problem that needs to be solved urgently in the current medical technology field.

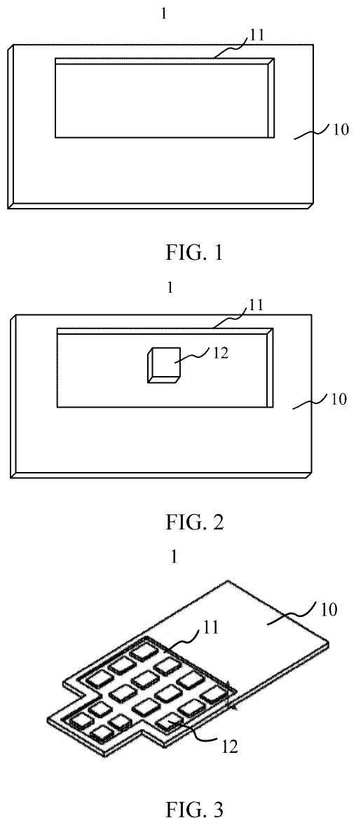

Referring to FIGS. 1-4, in some embodiments, an object positioning device 1 is provided. The object positioning device 1 includes at least one coil 14 and a positioning plate 10. At least a part of the at least one coil 14 is configured to be embedded in the positioning plate 10.

The positioning plate 10 is configured to, for example, accurately position the object, such as a patient's body, within a medical imaging device (e.g., magnetic resonance imaging scanner) and to stabilize the object during the scanning process, ensuring optimal imaging quality.

In the embodiments of the present disclosure, the object positioning device 1 includes the at least one coil 14 and the positioning plate 10, and the at least a part of the at least one coil 14 is configured to be embedded in the positioning plate 10. The at least one coil 14 in the object positioning device 1 and the positioning plate 10 are substantially in a same plane. As a non-limiting example of a method for embedding the coil 14 in the positioning plate 10, a groove may be optionally provided in the positioning plate 10, and the at least one coil 14 may be arranged in the groove. Alternatively, a slot may be provided in or on the positioning plate 10, and the at least a part of the coil 14 may be slidably arranged in the positioning plate 10 through the slot.

The object positioning device 1 in the embodiments of the present disclosure can not only receive and accommodate the at least one coil 14, but also enable the object to lie flat without being raised. The coil 14 in the embodiment of the present disclosure may be an RF receiving coil of any shape, and the RF receiving coil is configured to receive small magnetic resonance signals to perform magnetic resonance imaging on the object. The coil 14 may include a coil unit. Optionally, the coil 14 may be a coil with a hole, or a coil without a hole, which is not limited in the embodiments of the present disclosure.

The positioning plate 10 is configured to fix and support the at least one coil 14 to ensure that the coil 14 does not move or deform, or only moves or deforms within an allowable range. The positioning plate 10 in the embodiments of the present disclosure may be a positioning plate 10 of any shape. Exemplarily, the shape of the positioning plate 10 may include but is not limited to any one of a convex shape, a U shape, a groove shape, a rectangle, an ellipse, a trapezoid, a polygon, or the like. The U-shaped positioning plate 10 may be a U-shaped coil support structure bent upward on a periphery of the positioning plate, so as to increase the coverage of the coil 14 on the object, thereby improving the receiving efficiency of the RF receiving coil.

As described above, the object positioning device 1 includes the at least one coil 14 and the positioning plate 10, and the at least a part of the at least one coil 14 is configured to be embedded in the positioning plate 10. Since the at least a part of the at least one coil 14 is configured to be embedded in the positioning plate 10, the at least one coil 14 and the positioning plate 10 are in the same plane, and the positioning plate 10 can fix and support the coil 14. Accordingly, when the object positioning device 1 in the embodiments of the present disclosure is used for magnetic resonance imaging, the original design of the coil 14 does not need to be changed, i.e., other imaging performance aspects of the radiotherapy equipment will not be affected. Moreover, the object positioning device 1 can achieve the function of fixing and supporting the coil 14 without providing a positioning plate and a coil support pad above the coil 14. Therefore, the distance between the RF receiving coil and the body surface of the object can be shortened, thereby improving the receiving efficiency of the RF receiving coil without affecting other imaging performance aspects of the radiotherapy equipment.

In an embodiment, a groove 11 is provided on or in the positioning plate 10, and at least a part of the coil 14 is fixed in the groove 11, such that at least a part of the coil 14 is embedded in the positioning plate 10.

FIG. 1 is a schematic diagram illustrating a structure of an object positioning device in an embodiment. The object positioning device 1 includes at least one coil 14 (not shown in FIG. 1) and a positioning plate 10. A groove 11 is provided on or in the positioning plate 10, and at least a part of the coil 14 is fixed in the groove 11. Exemplarily, a depth of the groove 11 is less than or equal to a height of the positioning plate 10, and a height of the coil 14 is less than or equal to the depth of the groove 11. Optionally, a protrusion component 12 may be provided in the groove 11, so that the coil 14 is fixed through the protrusion component 12. Alternatively, a sliding component may be provided in the groove 11, so that the coil 14 is fixed by the sliding component. The configurations of the component that fixes the coil 14 are not limited in the embodiments of the present disclosure.

Regarding the design of the groove 11, optionally, a shape of the groove 11 may be preset, or the shape of the groove 11 may adapt to the shape of the positioning plate 10, so as to achieve a balance between the cost and the receiving efficiency of the RF receiving coil. Optionally, the groove 11 may be provided on the entire area of the positioning plate 10, or may be provided on a partial area of the positioning plate 10. The partial area is an area to be detected for magnetic resonance imaging using a magnetic resonance imaging device.

In an embodiment, the at least a part of the coil 14 is detachably fixed in the groove 11. It should be noted that in the embodiments of the present disclosure, optionally, the at least one coil 14 may be detachably fixed in the groove 11, or may be non-detachably fixed in the groove 11, which is not limited in the embodiments of the present disclosure.

In this embodiment, the groove 11 is provided on or in the positioning plate 10, and the at least a part of the coil 14 is fixed in the groove 11. Since the coil 14 is fixed in the groove 11 on the positioning plate 10, the coil 14 and the positioning plate 10 are in the same plane, and the positioning plate 10 can fix and support the coil 14. Accordingly, when the object positioning device 1 in the embodiments of the present disclosure is used for magnetic resonance imaging, the original design of the coil 14 does not need to be changed, i.e., other imaging performance aspects of the radiotherapy equipment will not be affected. Moreover, the object positioning device 1 can achieve the function of fixing and supporting the coil without providing a positioning plate and a coil support pad above the coil. Therefore, the distance between the RF receiving coil and the body surface of the object (e.g., a patient or an object to be scanned) can be shortened, thereby improving the receiving efficiency of the RF receiving coil without affecting other imaging performance aspects of the radiotherapy equipment.

In an embodiment, the positioning plate 10 further includes at least one protrusion component 12 disposed in the groove 11, and the at least a part of the coil 14 is arranged to surround the at least one protrusion component 12, which facilitates the fixing of the coil 14 in the groove 11.

In the embodiments of the present disclosure, the positioning plate 10 further includes the at least one protrusion component 12 disposed in the groove 11, and the at least a part of the coil 14 is arranged to surround the at least one protrusion component 12 to fix and support the coil 14, i.e., one or more protrusion components 12 are disposed in the groove 11 of the positioning plate 10, and correspondingly, one or more coils 14 may be fixed and supported by the one or more protrusion components 12, so that positions of the protrusion components 12 may be obtained, and positions of the coils 14 may be determined according to the positions of the protrusion components 12. Therefore, based on the position of the coil 14, high-precision modeling of the coil 14 can be achieved during the magnetic resonance imaging-guided radiotherapy.

In an embodiment, the at least a part of the coil 14 is arranged to detachably surround the protrusion component 12.

Since the at least a part of the coil 14 is wound around the protrusion component 12, and the coil 14 and the protrusion component 12 are designed to be detachable, the coil 14 may be removed from the protrusion component 12 when the coil at a certain position is not needed, thereby reducing the complexity of the operation for medical staff, and reducing the influence of the RF receiving coil at this position on magnetic resonance imaging. Optionally, the number of the protrusion components 12 in the groove 11 may be one or more, which is not limited in the embodiments of the present disclosure.

Exemplarily, as shown in FIG. 2 which illustrates a structure of an object positioning device including a protrusion component in an embodiment, the positioning plate 10 further includes a protrusion component 12 disposed in the groove 11, and the coil 14 (not shown in FIG. 2) is wound around the protrusion component 12.

In an embodiment, the positioning plate 10 includes a plurality of protrusion components 12, and the plurality of protrusion components 12 are disposed in the groove 11 in an array.

Exemplarily, as shown in FIG. 3 which illustrates a structure of an object positioning device 1 including a plurality of protrusion components 12 in an embodiment, the object positioning device 1 includes a plurality of coils 14 with holes (not shown in FIG. 3) and a convex-shaped positioning plate 10, and a groove 11 is provided in a partial area of the positioning plate 10. The plurality of coils 14 with holes correspond to the plurality of protrusion components 12 of the positioning plate 10. The plurality of protrusion components 12 are disposed in the groove 11 in a convex-shaped array, and the plurality of coils 14 with holes are correspondingly wound around the protrusion components 12. It can be understood that the hole of each coil 14 may be tightly fitted with the protrusion component(s) 12 corresponding to the coil 14. Optionally, one coil 14 may be wound around one protrusion component 12 corresponding to the coil 14, or one coil 14 may be wound around a plurality of protrusion components 12 corresponding to the coil 14, which is not limited in the embodiments of the present disclosure. In addition, the embodiments of the present disclosure do not limit the arrangement of the plurality of protrusion components 12. Exemplarily, as shown in FIG. 3, the plurality of protrusion components 12 may be arranged in a convex-shaped array. Alternatively, the plurality of protrusion components 12 may be arranged in a rectangular array. The embodiments of the present disclosure do not limit the position of the groove 11. Exemplarily, as shown in FIG. 3, the groove 11 may be provided on a partial area of the positioning plate 10, and the partial area is an area to be detected for magnetic resonance imaging using a magnetic resonance imaging device. Alternatively, the groove 11 may be provided on the entire area of the positioning plate 10, i.e., the positioning plate 10 is of a groove structure.

In an exemplary embodiment, a shape of the protrusion component 12 may include but is not limited to any of a rectangle, a square, a circle, an ellipse, a triangle, a rhombus, a trapezoid, a parallelogram, a polygon, or the like.

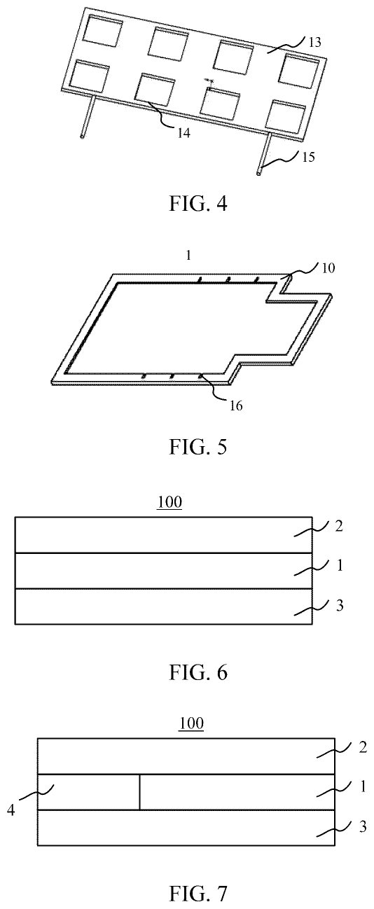

FIG. 4 is a schematic diagram illustrating a structure of a plurality of coils 14 with holes in an embodiment. Refereeing to FIGS. 3-4, a coil plate 13 includes a plurality of coils 14 with holes, and the plurality of coils 14 with holes are arranged in a rectangular array. The hole of each coil 14 can be tightly fitted with the corresponding protrusion component 12 in FIG. 3. In addition, the coil plate 13 further includes signal transmission lines 15 for the coils 14, and the signal transmission lines 15 are connected to the coils 14 for transmitting signals of the coil 14. The coil 14 may be an RF receiving coil.

In this embodiment, the positioning plate 10 further includes the at least one protrusion component 12 disposed in the groove 11, and the coil 14 is arranged to surround the protrusion component 12. This embodiment of the present disclosure is applicable to the coil 14 with a hole, and the coil 14 may be fixed and supported by winding the coil 14 around the protrusion component 12. Accordingly, when the object positioning device 1 in the embodiments of the present disclosure is used for magnetic resonance imaging, the original design of the coil 14 does not need to be changed, i.e., other imaging performance aspects of the radiotherapy equipment will not be affected. Moreover, the object positioning device 1 can achieve the function of fixing and supporting the coil without the need to provide a positioning plate and a coil support pad on the object positioning device 1. Therefore, the distance between the RF receiving coil and the body surface of the object can be shortened, thereby improving the receiving efficiency of the RF receiving coil without affecting other imaging performance aspects of the radiotherapy equipment.

In another embodiment, as shown in FIG. 5 which illustrates an exemplary structure of an object positioning device 1, the positioning plate 10 further includes sliding components 16. The sliding components 16 are provided on any two opposite sides of the groove 11, and the sliding components 16 on both sides are connected to two ends of the coil 14, respectively.

In the embodiments of the present disclosure, the object positioning device 1 further includes at least one sliding component 16. The sliding components 16 are provided on any two opposite sides of the groove 11, and the sliding components 16 on both sides are connected to two ends of the coil 14, respectively, so as to fix and support the coil 14. Since the sliding components 16 on both sides are connected to the two ends of the coil 14, respectively, the coil 14 may slide on the sliding components 16, and may even be removed from the sliding components 16, so that a relative position of the coil 14 in the object positioning device 1 may be changed. Therefore, the coils can be more accurately positioned in the area to be detected for magnetic resonance imaging, thereby performing more accurate magnetic resonance imaging on the area to be detected. In addition, the coil 14 and the sliding components 16 are designed to be detachable, so that the coil 14 may be removed from the sliding components 16 when the coil 14 at a certain position is not used, thereby reducing the complexity of the operation for medical staff, and reducing the influence of the RF receiving coil at this position on magnetic resonance imaging.

The positioning plate 10 further includes sliding components 16, and a groove 11 may be provided on the entire area of the positioning plate 10, i.e., the positioning plate 10 has a groove structure. A plurality of sliding components 16 are provided on opposite sides of the groove 11, and the sliding components 16 on the opposite sides of the groove 11 are connected to two ends of the coil 14 (not shown in FIG. 5), respectively. Optionally, the coil 14 may be a coil with a hole, or the coil 14 may be a coil without a hole, which is not limited in the embodiments of the present disclosure.

In this embodiment, the positioning plate 10 further includes the sliding components 16. The sliding components 16 are provided on any two opposite sides of the groove 11, and the sliding components 16 on both sides are connected to the two ends of the coil 14, respectively. This embodiment of the present disclosure is applicable to the coil with a hole or the coil without a hole, and the coil 14 may be fixed and supported by slidably fixing the coil 14 on the sliding component 16. Accordingly, when the object positioning device 1 in the embodiments of the present disclosure is used for magnetic resonance imaging, the original design of the coil 14 does not need to be changed, i.e., other imaging performance aspects of the radiotherapy equipment will not be affected. Moreover, the object positioning device 1 can achieve the function of fixing and supporting the coil without the need to provide a positioning plate and a coil support pad above the object positioning device 1. Therefore, the distance between the RF receiving coil and the body surface of the object can be shortened, so that the signal-to-noise ratio can be improved under the design of multi-channel, small-sized RF receiving coils, without affecting other imaging performance aspects of the radiotherapy equipment thereby improving the receiving efficiency of the RF receiving coil to obtain a higher quality magnetic resonance image.

Referring to FIG. 6 which illustrates a structure of a bed 100, in an embodiment, a bed 100 is provided for supporting a scanned object. The bed 100 includes the object positioning device 1 of any one of the above embodiments, an isolation pad 2, and a bed body 3. The object positioning device 1 is arranged on the bed body 3, and the isolation pad 2 is arranged on the object positioning device 1.

The bed 100 includes at least one object positioning device 1, an isolation pad 2, and a bed body 3. The object positioning device 1 is arranged on the bed body 3, and the isolation pad 2 is arranged on the object positioning device 1. Optionally, there may be a gap between the object positioning device 1, the isolation pad 2 and the bed body 3, or there may be no gap between the object positioning device 1, the isolation pad 2 and the bed body 3. In addition, optionally, a width of the object positioning device 1 may be equal to a width of the bed body 3, or the width of the object positioning device 1 may be less than the width of the bed body 3, which is not limited in the embodiments of the present disclosure. The bed 100 is configured to support the scanned object. A scanning process may include but is not limited to a magnetic resonance (MR) imaging scanning process, a positron emission tomography-magnetic resonance (PET-MR) imaging scanning process, and the like. The isolation pad 2 is configured to, for example, assist an object (e.g., a patient or an object to be scanned) in positioning and/or prevent the conduction or radiation of heat. The isolation pad 2 may include, for example, a vacuum pad, a heat-insulating pad, etc. The bed body 3 is configured to support the object positioning device 1 and the isolation pad 2. It should be noted that, since the bed 100 may include the at least one object positioning device 1, in a case that the bed 100 includes a plurality of object positioning devices 1, the object positioning devices 1 may be fixed after being spliced.

In an embodiment, the object positioning device 1 includes at least one coil 14 and a positioning plate 10. At least a part of the at least one coil 14 is configured to be embedded in the positioning plate 10.

In an embodiment, a groove 11 is provided on or in the positioning plate 10, and the at least a part of the at least one coil 14 is fixed in the groove 11.

In an embodiment, a depth of the groove 11 is less than or equal to a height of the positioning plate 10, and a height of the at least one coil 14 is less than or equal to the depth of the groove 11.

In an embodiment, a slot is provided on or in the positioning plate 10, and at least a part of the at least one coil 14 is slidably arranged in the positioning plate 10 through the slot.

In an embodiment, the positioning plate 10 further includes at least one protrusion component 12 disposed in the groove 11, and the at least a part of the at least one coil 14 is arranged to surround the at least one protrusion component 12.

In an embodiment, the positioning plate 10 includes a plurality of protrusion components 12, and the plurality of protrusion components 12 are disposed in the groove 11 in an array.

In an embodiment, the at least a part of the at least one coil 14 is arranged to detachably surround the at least one protrusion component 12.

In an embodiment, the at least a part of the at least one coil 14 is detachably fixed in the groove 11.

In an embodiment, the positioning plate 10 further includes sliding components 16. The sliding components 16 are provided on any two opposite sides of the groove 11, and the sliding components 16 on both sides are connected to two ends of the coil 14, respectively.

In an embodiment, the at least one coil 14 includes an RF receiving coil.

In an embodiment, the positioning plate 10 includes a U-shaped coil support structure bent upward on a periphery of the positioning plate 10.

In an embodiment, the object positioning device 1 further includes a coil plate 13. The coil plate 13 is provided with a plurality of coils 14 with holes, and the plurality of coils with holes 14 are arranged in an array. The hole of each coil 14 is configured to snap with a corresponding protrusion component 12.

In an embodiment, the coil plate 13 is further provided with signal transmission lines 15 for the coils 14, and signal transmission lines 15 are configured to transmit signals of the coils 14.

In the above bed 100 configured to support the scanned object, the bed 100 includes the at least one object positioning device 1, the isolation pad 2 and the bed body 3. The object positioning device 1 is arranged on the bed body 3, and the isolation pad 2 is arranged on the object positioning device 1. Since the bed 100 in the embodiments of the present disclosure includes the at least one object positioning device 1, and the at least a part of the at least one coil 14 is configured to be embedded in the positioning plate 10, the at least one coil 14 and the positioning plate 10 are in the same plane, and the positioning plate 10 can fix and support the coil 14. Accordingly, when the object positioning device 1 in the embodiments of the present disclosure is used for magnetic resonance imaging, the original design of the coil 14 does not need to be changed, i.e., other imaging performance aspects of the radiotherapy equipment will not be affected. Moreover, the object positioning device 1 can achieve the function of fixing and supporting the coil without the need to provide a positioning plate and a coil support pad above the object positioning device 1. Therefore, the distance between the RF receiving coil and the body surface of the object can be shortened, thereby improving the receiving efficiency of the RF receiving coil without affecting other imaging performance aspects of the radiotherapy equipment.

In an embodiment, the bed 100 further includes at least one support pad 4. The at least one support pad 4 and the object positioning device 1 are arranged substantially in a same plane, and the isolation pad 2 is arranged on the object positioning device 1 and the at least one support pad 4.

FIG. 7 is a schematic diagram illustrating an exemplary structure ofa bed 100 including a support pad in an embodiment. As shown in FIG. 7, in the embodiments of the present disclosure, the bed 100 further includes the at least one support pad 4. The support pad 4 and the object positioning device 1 are arranged substantially in a same horizontal plane. In this case, the width of the object positioning device 1 is less than the width of the bed body 3, a sum of the widths of the support pad 4 and the object positioning device 1 is equal to the width of the bed body 3, and the width of the bed body 3 is equal to a width of the isolation pad 2. The isolation pad 2 is arranged on the object positioning device 1 and the support pad 4. Optionally, there may be a gap between the support pad 4, the object positioning device 1, the isolation pad 2 and the bed body 3, or there may be no gap between the support pad 4, the object positioning device 1, the isolation pad 2 and the bed body 3. The embodiments of the present disclosure do not limit the number of support pads 4 and the number of object positioning devices 1.

The bed 100 includes an object positioning device 1, an isolation pad 2, a bed body 3 and a support pad 4. The support pad 4 and the object positioning device 1 are arranged in the same horizontal plane. The object positioning device 1 and the support pad 4 are each arranged on an upper surface of the bed body 3, and the isolation pad 2 is arranged on upper surfaces of the object positioning device 1 and the support pad 4. The object may be located on an upper surface of the isolation pad 2. A sum of the widths of the support pad and the object positioning device 1 is equal to the width of the bed body 3, and the width of the bed body 3 is equal to the width of the isolation pad 2.

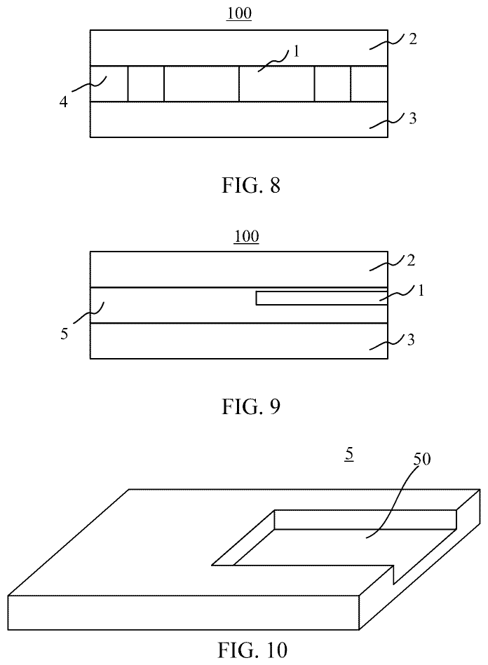

In another exemplary embodiment, as shown in FIG. 8 which illustrates a structure of a bed body 100 including a plurality of support pads in an embodiment, the bed body 100 includes a plurality of object positioning devices 1, an isolation pad 2, a bed body 3 and a plurality of support pads 4. The support pads 4 and the object positioning devices 1 are arranged substantially in a same horizontal plane. The object positioning devices 1 and the support pads 4 are each arranged on an upper surface of the bed body 3, and the isolation pad 2 is arranged on upper surfaces of the object positioning devices 1 and the support pads 4. The object may be located on an upper surface of the isolation pad 2. A sum of a total width of the plurality of support pads 4 and a total width of the plurality of object positioning devices 1 is equal to the width of the bed body 3, and the width of the bed body 3 is equal to the width of the isolation pad 2. The plurality of object positioning devices 1 and the plurality of support pads 4 may be designed to be detachable, thereby improving the flexibility of placing the object positioning devices 1.

In this embodiment, the bed 100 further includes the at least one support pad 4, the support pad 4 and the object positioning device 1 are arranged on the same plane, and the isolation pad 2 is arranged on the object positioning device 1 and the support pad 4. Through the detachable design of the at least one support pad 4 and the at least one object positioning device 1, the distance between the RF receiving coil and the body surface of the object can be shortened, thereby improving the receiving efficiency of the RF receiving coil without affecting other imaging performance aspects of the radiotherapy equipment. In addition, the flexibility of placing the object positioning device 1 can be improved. Furthermore, by arranging the at least one support pad 4 and the at least one object positioning device 1 that have the substantially same height in the same horizontal plane, the support pad 4 and the object positioning device 1 may jointly provide a flat surface for supporting and positioning the object, thereby improving the quality of a received signal while ensuring the comfort of the object.

As shown in FIG. 9 which illustrates a structure ofa bed 100 including a bed board 5, in an embodiment, the bed 100 further includes a bed board 5. The object positioning device 1 is embedded in the bed board 5, and the bed board 5 is arranged on the bed body 3. As shown in FIG. 10, the bed board 5 has a concave 50, for example, recessed downward from a top surface of the bed board 5, and the object positioning device 1 is embedded within the concave 50.

In this embodiment of the present disclosure, the bed 100 includes an object positioning device 1, an isolation pad 2, a bed body 3 and a bed board 5. The object positioning device 1 is embedded in the bed board 5. The bed board 5 is arranged on an upper surface of the bed body 3, and the isolation pad 2 is arranged on an upper surface of the bed board 5. The object may be located on an upper surface of the isolation pad 2. A width of the bed board 5 is equal to a width of the bed body 3, and the width of the bed body 3 is equal to a width of the isolation pad 2.

In addition, in another embodiment, the bed board 5 may be arranged on the upper surface of the bed body 3, the object positioning device 1 is arranged on the upper surface of the bed board 5, and the isolation pad 2 is arranged on an upper surface of the object positioning device 1. The object may be located on the upper surface of the isolation pad 2. The specific position of the bed board 5 may be arranged according to actual needs, which is not limited in the embodiments of the present disclosure.

In this embodiment, the bed 100 further includes the bed board 5, the object positioning device 1 is embedded in the bed board 5, and the bed board 5 is arranged on the upper surface of the bed body 3. By designing that the object positioning device 1 is embedded in the bed board 5, the distance between the RF receiving coil and the body surface of the object can be shortened, thereby improving the receiving efficiency of the RF receiving coil without affecting other imaging performance aspects of the radiotherapy equipment. In addition, the object positioning device 1 can be accurately arranged in the area to be detected for magnetic resonance imaging, thereby reducing the design cost of the object positioning device 1.

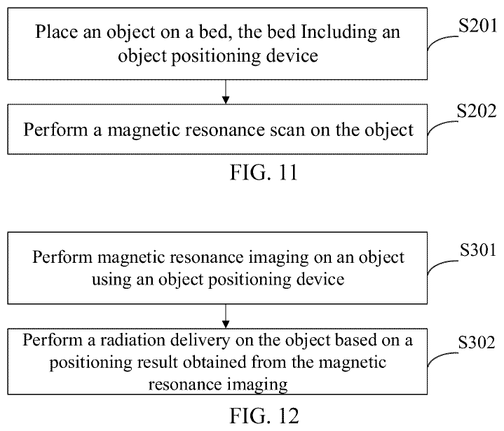

In an embodiment, as shown in FIG. 11, a magnetic resonance scanning method is provided. Taking the method applied to a magnetic resonance scanning device as an example for illustration, the method includes the following steps.

In step S201, an object is placed on a bed 100. The bed 100 includes the object positioning device 1 of any one of the above embodiments.

In step S202, a magnetic resonance scan is performed on the object.

The bed 100 includes any one of the beds 100 described in the above embodiments, and the bed 100 includes the object positioning device 1 of any one of the above embodiments. The object refers to an object for whom a magnetic resonance scan is to be performed, such as a patient or an object to be scanned.

In the embodiments of the present disclosure, the object may be placed on the bed 100 firstly. After determining that the object is correctly placed, a magnetic resonance scan may be performed on the object. Exemplarily, the basis for determining that the object is correctly placed may include determining that the object is placed at a designated position on the bed 100 and the object is fixed on the bed 100. The designated position on the bed 100 may be directly above the object positioning device 1, where the receiving efficiency of the RF receiving coil is relatively high.

In the magnetic resonance scanning method described above, the object is placed on the bed 100, and the bed 100 includes the object positioning device 1 of any one of the above embodiments. Then, the magnetic resonance scanning is performed on the object. Since the bed 100 in this embodiment of the present disclosure includes the at least one object positioning device 1, and at least a part of at least one coil 14 in the object positioning device 1 is configured to be embedded in the positioning plate 10, the at least one coil 14 and the positioning plate 10 are in the same plane, and the positioning plate 10 can fix and support the coil 14. Accordingly, when the object is placed on the bed 100 in the embodiments of the present disclosure and the magnetic resonance scanning is performed on the object, the original design of the coil 14 does not need to be changed, i.e., other imaging performance aspects of the radiotherapy equipment will not be affected. Moreover, the object positioning device 1 can achieve the function of fixing and supporting the coil without the need to provide a positioning plate and a coil support pad on the object positioning device 1. Therefore, the distance between the RF receiving coil and the body surface of the object can be shortened, thereby improving the receiving efficiency of the RF receiving coil without affecting other imaging performance aspects of the radiotherapy equipment.

In an embodiment, as shown in FIG. 12, a radiation delivery method is provided. Taking the method applied to a magnetic resonance scanning device or a radiotherapy device (or a device integrating the functions of magnetic resonance imaging and radiotherapy) as an example for illustration, the method includes the following steps.

In step S301, magnetic resonance imaging is performed on an object using an object positioning device 1 of any one of the above embodiments.

In step S302, a radiation delivery is performed on the object based on a positioning result obtained from the magnetic resonance imaging.

In this embodiment of the present disclosure, magnetic resonance imaging is performed on the object using the object positioning device 1 of any one of the above embodiments, so as to obtain the positioning result of the magnetic resonance imaging. Further, based on the positioning result of the magnetic resonance imaging, it can be determined whether the positioning result obtained from the magnetic resonance imaging is accurate. If the positioning result is accurate, it means that the object is correctly placed. In this case, the radiation delivery may be performed on the object. If the positioning result is not accurate, it means that the object is incorrectly placed. In this case, the placement position of the object needs to be changed. After determining that the object is correctly placed, the radiation delivery may be performed on the object.

As a non-limiting example, the positioning plate 10 may be used for magnetic resonance simulation positioning of a patient before radiotherapy (i.e., magnetic resonance imaging is performed on a specific body part of the patient (such as a target volume) before radiotherapy to determine a shape and a position of the specific body part of the patient), or the positioning plate 10 may be used for real-time magnetic resonance-guided treatment of the patient. In the embodiments of the present disclosure, the positioning plate 10 may be used to support the patient for magnetic resonance scanning, so as to obtain an image (i.e., the positioning result of the magnetic resonance imaging) obtained from the magnetic resonance scanning, and then a radiotherapy plan is formulated or radiotherapy is guided based on the image obtained by the magnetic resonance scanning.

The bed 100 is the bed 100 of any one of the above embodiments, and the bed 100 includes the object positioning device 1 of any one of the above embodiments. The object refers to an object that needs a magnetic resonance scan, such as a patient or an object to be scanned. A radiation delivery process may include but is not limited to a radiation therapy process, a radiation processing process, a radiation flaw detection process, a radiation test process, or the like. In addition, the radiation delivery process may be fully automatic.

In the above radiation delivery method, the magnetic resonance imaging is performed on the object using the object positioning device 1 of any one of the above embodiments, and then the radiation delivery is performed on the object based on the positioning result obtained from the magnetic resonance imaging. Since the at least a part of the at least one coil 14 in the object positioning device 1 in the embodiments of the present disclosure is configured to be embedded in the positioning plate 10, the at least one coil 14 and the positioning plate 10 are in the same plane, and the positioning plate 10 can fix and support the coil 14. Accordingly, when the magnetic resonance imaging is performed on the object using the object positioning device 1 of any one of the above embodiments, and then the radiation delivery is performed on the object based on the positioning result obtained from the magnetic resonance imaging, the original design of the coil 14 does not need to be changed, i.e., other imaging performance aspects of the radiotherapy equipment will not be affected. Moreover, the object positioning device 1 can achieve the function of fixing and supporting the coil without the need to provide a positioning plate and a coil support pad above it. Therefore, the distance between the RF receiving coil and the body surface of the object can be shortened, thereby improving the receiving efficiency of the RF receiving coil without affecting other imaging performance aspects of the radiotherapy equipment.

The technical features in the above embodiments may be combined arbitrarily. For concise description, not all possible combinations of the technical features in the above embodiments are described. However, provided that they do not conflict with each other, all combinations of the technical features are to be considered to be within the scope described in this specification.

The above-mentioned embodiments only describe several implementations of the present disclosure, and their description is specific and detailed, but should not be understood as a limitation on the patent scope of the present disclosure. It should be noted that, for a person of ordinary skill in the art may further make variations and improvements without departing from the conception of the present disclosure, and these all fall within the protection scope of the present disclosure. Therefore, the protection scope of the present disclosure should be subject to the appended claims.

What is claimed is:

1. An object positioning device, comprising at least one coil and a positioning plate, wherein at least a part of the at least one coil is configured to be embedded in the positioning plate.

2. The object positioning device according to claim 1, wherein a groove is provided on or in the positioning plate, and the at least a part of the at least one coil is fixed in the groove.

3. The object positioning device according to claim 2, wherein a depth of the groove is less than or equal to a height of the positioning plate, and a height of the at least one coil is less than or equal to the depth of the groove.

4. The object positioning device according to claim 1, wherein a slot is provided on or in the positioning plate, and the at least a part of the at least one coil is slidably arranged in the positioning plate through the slot.

5. The object positioning device according to claim 2, wherein the positioning plate further comprises at least one protrusion component (12) disposed in the groove, and the at least a part of the at least one coil is arranged to surround the at least one protrusion component.

6. The object positioning device according to claim 5, wherein the positioning plate comprises a plurality of protrusion components, and the plurality of protrusion components are disposed in the groove in an array.

7. The object positioning device according to claim 5, wherein the at least a part of the at least one coil is arranged to detachably surround the at least one protrusion component.

8. The object positioning device according to claim 2, wherein the at least a part of the at least one coil is detachably fixed in the groove.

9. The object positioning device according to claim 2, wherein the positioning plate further comprises sliding components, the sliding components are provided on any two opposite sides of the groove, and the sliding components on both sides are connected to two ends of the at least one coil, respectively.

10. The object positioning device according to claim 1, wherein the at least one coil comprises a radio frequency receiving coil.

11. The object positioning device according to claim 1, wherein the positioning plate comprises a U-shaped coil support structure bent upward on a periphery of the positioning plate.

12. The object positioning device according to claim 8, further comprising a coil plate, wherein the coil plate is provided with a plurality of coils with holes, the plurality of coils are arranged in an array, and the hole of each coil is configured to snap with the protrusion component correspondingly.

13. The object positioning device according to claim 12, wherein the coil plate is further provided with signal transmission lines for the coils, and the signal transmission lines are configured to transmit signals of the coils.

14. A bed, comprising an object positioning device, an isolation pad and a bed body, the object positioning device being arranged on the bed body, the isolation pad being arranged on the object positioning device,
wherein the object positioning device comprises at least one coil and a positioning plate, and at least a part of the at least one coil is configured to be embedded in the positioning plate.

15. The bed according to claim 14, wherein the bed further comprises at least one support pad, the at least one support pad and the object positioning device are arranged substantially in a same plane, the at least one support pad is arranged on the bed body, and the isolation pad is arranged on the object positioning device and the at least one support pad.

16. The bed according to claim 14, wherein the bed further comprises a bed board, the object positioning device is embedded in the bed board, the bed board is arranged on the bed body, and the isolation pad is arranged on the bed board.

17. The bed according to claim 16, wherein the bed board has a concave, and the object positioning device is embedded within the concave.

18. The bed according to claim 14, wherein the bed further comprises a bed board, the bed board is arranged on the bed body, the object positioning device is arranged on the bed board, and the isolation pad is arranged on the object positioning device.

19. The bed according to claim 14, wherein a groove is provided on or in the positioning plate, the at least a part of the at least one coil is fixed in the groove; and
wherein the positioning plate further comprises at least one protrusion component disposed in the groove, and the at least a part of the at least one coil is arranged to surround the at least one protrusion component.

20. A magnetic resonance scanning method, comprising:
placing an object on a bed, the bed comprising an object positioning device, an isolation pad and a bed body, the object positioning device being arranged on the bed body, and the isolation pad being arranged on the object positioning device, the object positioning device comprising at least one coil and a positioning plate, and at least a part of the at least one coil being configured to be embedded in the positioning plate; and
performing a magnetic resonance scan on the object.
